(19) United States
(12) Patent Application Publication
(10) Pub. No.: US 2024/0389936 A1
(43) Pub. Date: Nov. 28, 2024

(54) HEMODYNAMIC MONITOR WITH NOCICEPTION DETECTION

(71) Applicant: EDWARDS LIFESCIENCES CORPORATION, Irvine, CA (US)

(72) Inventors: Zhongping Jian, Irvine, CA (US); Jacobus Jozef Gerardus Maria Settels, De Hoef (NL); Feras Al Hatib, Irvine, CA (US); Christine Lee, Long Beach, CA (US)

(21) Appl. No.: 18/794,938

(22) Filed: Aug. 5, 2024

Related U.S. Application Data
(63) Continuation of application No. PCT/US2023/012364, filed on Feb. 5, 2023.
(60) Provisional application No. 63/307,240, filed on Feb. 7, 2022.

(51) Int. Cl. A61B 5/00; A61B 5/0205; A61B 5/021

(52) U.S. Cl. CPC A61B 5/4824; A61B 5/0205; A61B 5/6826; A61B 5/7235; A61B 5/7405; A61B 5/742; A61B 5/746; A61B 5/021

(57) ABSTRACT

A method for monitoring arterial pressure of a patient and detecting nociception of the patient includes receiving, by a hemodynamic monitor, sensed hemodynamic data representative of an arterial pressure waveform of the patient. A hardware processor of the hemodynamic monitor performs waveform analysis of the sensed hemodynamic data to calculate a plurality of signal measures of the sensed hemodynamic data. The hardware processor of the hemodynamic monitor calculates cross-correlational association measurements between each of the signal measures. The cross-correlational association measurements are outputted to a user interface. The cross-correlational association measurements are monitored for bursts in the cross-correlational association measurements. A nociception event of the patient is detected when a burst in one or more of the cross-correlational association measurements is outputted to the user interface.

### CROSS-REFERENCE TO RELATED APPLICATION(S)

This application claims the benefit of International Application No. PCT/US2023/012364, filed Feb. 5, 2023, and entitled “HEMODYNAMIC MONITOR WITH NOCICEPTION DETECTION,” the disclosure of which is hereby incorporated by reference in its International Application No. PCT/US2023/012364 claims the benefit of U.S. entirety. Provisional Application No. 63/307,240, filed Feb. 7, 2022, and entitled “HEMODYNAMIC MONITOR WITH NOCICEPTION DETECTION,” the disclosure of which is hereby incorporated by reference in its entirety.

### BACKGROUND

The present disclosure relates generally to hemodynamic monitoring, and in particular, to detecting and predicting nociception in a patient using monitored hemodynamic data. Nociception is the process in which nerve endings called nociceptors detect noxious stimuli and send a signal to the central nervous system which is interpreted as pain. Noxious insult initiates a “sharp” signal from the source of the insult. Locally, nociception initiates an inflammation response. The signal then travels through neurons to the spinal column where a muscle reflex is triggered. The signal continues to the brain, where, upon reaching the lower brain, the nociception signal triggers a sympathetic nervous system response. Nociception can cause a sympathetic nervous system response without reaching consciousness or before reaching consciousness; thus, an unconscious patient in surgery or in intensive care can experience pain. To prevent a patient from awaking out of surgery or intensive care in pain, medical workers administer analgesics to the patient before and/or during surgery and at various times in the intensive care. However, knowing the amount of analgesic to administer can be difficult as pain thresholds and tolerances vary from patient to patient, and the patient is unable to verbally communicate or signal feedback while unconscious. Administering too little analgesic to the patient during surgery can result in the patient awaking in pain after the surgery. Administering too much analgesic to the patient during surgery can result in the patient experiencing nausea, drowsiness, impaired thinking skills, and impaired function.

In view of the negative consequences of administering too little analgesic to the patient and the negative consequences of administering too much analgesic to the patient, a solution is needed that will allow medical workers the ability to detect or predict nociception of an unconscious patient during surgery. Accurately detecting or predicting nociception of a patient during surgery can help medical workers know the appropriate amount of analgesic to administer to the patient so that the patient does not awake from surgery with significant pain, without providing too much analgesic to the patient.

### SUMMARY

In one example, a method for monitoring arterial pressure of a patient and detecting nociception of the patient, includes receiving, by a hemodynamic monitor, sensed hemodynamic data representative of an arterial pressure waveform of the patient from a hemodynamic sensor. The method further includes performing, by a hardware processor of the hemodynamic monitor, waveform analysis of the sensed hemodynamic data to calculate a plurality of signal measures of the sensed hemodynamic data. The hardware processor of the hemodynamic monitor organizes the plurality of signal measures into pairs of signal measures. The hardware processor of the hemodynamic monitor measures a cross-correlational association of each of the pairs of signal measures. A measured cross-correlational association of each of the pairs of signal measures is outputted to a display. The measured cross-correlational association of each of the pairs of signal measures is monitored for an increase above a predetermined threshold in the measured cross-correlational association of at least one of the pairs of signal measures. The hemodynamic monitor sends an alert to medical personnel of a nociception event of the patient when the measured cross-correlational associate of at least one of the pairs of signal measures increases above the predetermined threshold.

In another example, a method for monitoring arterial pressure of a patient and detecting nociception of the patient includes receiving, by a hemodynamic monitor, sensed hemodynamic data representative of an arterial pressure waveform of the patient. A hardware processor of the hemodynamic monitor performs waveform analysis of the sensed hemodynamic data to calculate a plurality of signal measures of the sensed hemodynamic data. The hardware processor of the hemodynamic monitor calculates cross-correlational association between each of the signal measures. The cross-correlational association measurements are outputted to a user interface. The cross-correlational association measurements are monitored for bursts in the cross-correlational association measurements. A nociception event of the patient is detected when a burst in one or more of the cross-correlational association is outputted to the user interface.

In another example, a system for monitoring arterial pressure of a patient and providing a warning to medical personnel of nociception of the patient includes a hemodynamic sensor that produces hemodynamic data representative of an arterial pressure waveform of the patient. The system further includes a system memory that stores nociception detection software code and a user interface that includes a sensory alarm that provides a sensory signal to warn the medical personnel of a nociception event of the patient. A hardware processor is configured to execute the nociception detection software code to perform waveform analysis of the hemodynamic data to determine a plurality of signal measures. The plurality of signal measures is organized into pairs of signal measures and a cross-correlational association of each of the pairs of signal measures is measured. Measurements of cross-correlational association of the pairs of signal measures are outputted to the user interface. The measurements of cross-correlational association of the pairs of signal measures are monitored for bursts in the measurements of cross-correlational association of the pairs of signal measures. A sensory alarm of the user interface is invoked, indicating a nociception event in response to a burst in one or more of the measurements of cross-correlational association of the pairs of signal measures.

### BRIEF DESCRIPTION OF THE DRAWINGS

FIG. 1 is a perspective view of an example hemodynamic monitor that analyzes an arterial pressure of a patient and provides a warning to medical personnel of a nociception event of the patient.

FIG. 2 is a perspective view of an example minimally invasive pressure sensor for sensing hemodynamic data representative of arterial pressure of a patient.

FIG. 3 is a perspective view of an example non-invasive sensor for sensing hemodynamic data representative of arterial pressure of a patient.

FIG. 4 is a block diagram illustrating an example hemodynamic monitoring system that detects a current nociception event for a patient based on the arterial pressure of the patient.

FIG. 5 is a graph illustrating an example trace of an arterial pressure waveform including example indicia corresponding to signal measures used by hemodynamic monitoring system to detect a current nociception event of the patient.

FIG. 6 is a graph illustrating a systolic blood pressure, a heart rate, and a collection of cross-correlational association measure plots related to the systolic blood pressure and the heart rate all plotted over time.

### DETAILED DESCRIPTION

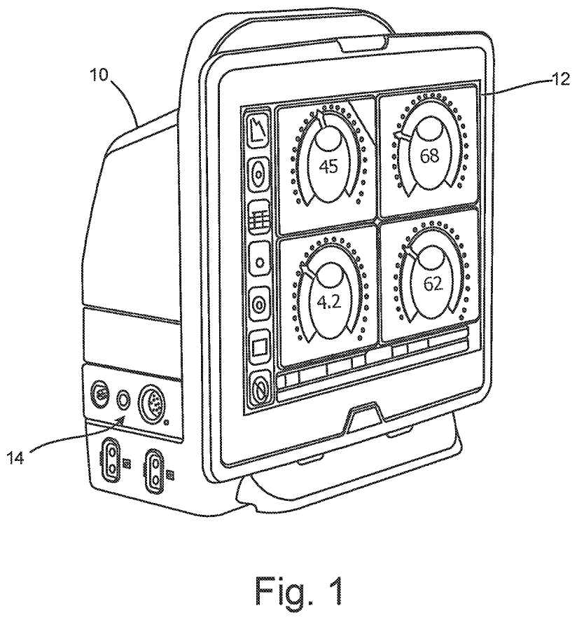

As described herein, a hemodynamic monitoring system senses hemodynamic data from a patient and uses the hemodynamic data to detect whether the patient is experiencing a current nociception event. The hemodynamic monitoring system is able to detect a current nociception event from the hemodynamic data of the patient by deriving signal measures from the hemodynamic data, organizing the signal measures into pairs, calculating a cross-correlational association for each of the pairs of signal measures, and monitoring the cross-correlational association for each of the pairs of signal measures. When a burst in value occurs in at least one of the cross-correlational association of the pairs of signal measures, the hemodynamic monitoring system can raise a signal or an alarm to medical workers to alert the medical workers that the patient is experiencing a current nociception event. After receiving the signal, the medical workers can administer an analgesic to the patient to mitigate the nociception event. The hemodynamic monitoring system is described in detail below with reference to FIGS. 1-6.

FIG. 1 is a perspective view of hemodynamic monitor 10 that detects a nociception event of a patient. As illustrated in FIG. 1, hemodynamic monitor 10 includes display 12 that, in the example of FIG. 1, presents a graphical user interface including control elements (e.g., graphical control elements) that enable user interaction with hemodynamic monitor 10. Hemodynamic monitor 10 can also include a plurality of input and/or output (I/O) connectors configured for wired connection (e.g., electrical and/or communicative connection) with one or more peripheral components, such as one or more hemodynamic sensors, as is further described below. For instance, as illustrated in FIG. 1, hemodynamic monitor 10 can include I/O connectors 14. While the example of FIG. 1 illustrates five separate I/O connectors 14, it should be understood that in other examples, hemodynamic monitor 10 can include fewer than five I/O connectors or greater than five I/O connectors. In yet other examples, hemodynamic monitor 10 may not include I/O connectors 14, but rather may communicate wirelessly with various peripheral devices.

As further described below, hemodynamic monitor 10 includes one or more processors and computer-readable memory that stores nociception detection software code which is executable to produce a score representing a probability of a present (i.e., current) nociception event for a patient. Hemodynamic monitor 10 can receive sensed hemodynamic data representative of an arterial pressure waveform of the patient, such as via one or more hemodynamic sensors connected to hemodynamic monitor 10 via I/O connectors 14. Hemodynamic monitor 10 executes the nociception detection software code to obtain, using the received hemodynamic data, a plurality of signal measures, which can include one or more vital sign parameters characterizing vital sign data of the patient as is further described below.

As illustrated in FIG. 1, hemodynamic monitor 10 can present a graphical user interface at display 12. Display 12 can be a liquid crystal display (LCD), a light-emitting diode (LED) display, an organic light-emitting diode (OLED) display, or other display device suitable for providing information to users in graphical form. In some examples, such as the example of FIG. 1, display 12 can be a touch-sensitive and/or presence-sensitive display device configured to receive user input in the form of gestures, such as touch gestures, scroll gestures, zoom gestures, swipe gestures, or other gesture input.

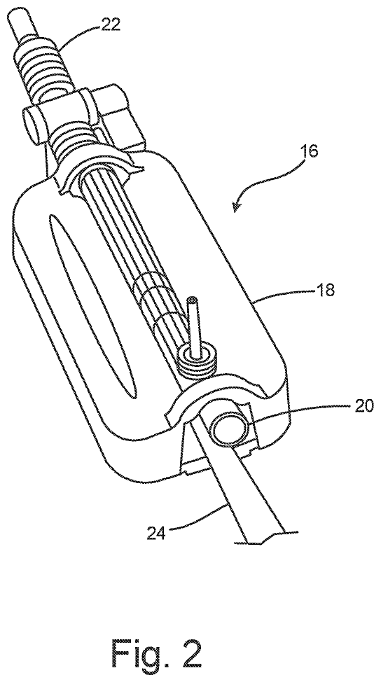

FIG. 2 is a perspective view of hemodynamic sensor 16 that can be attached to a patient for sensing hemodynamic data representative of arterial pressure of the patient. Hemodynamic sensor 16, illustrated in FIG. 2, is one example of a minimally invasive hemodynamic sensor that can be attached to the patient via, e.g., a fluid-filled tubing connected to a radial arterial catheter inserted into an arm of the patient and to which hemodynamic sensor 16 is attached. In other examples, hemodynamic sensor 16 can be attached to the patient via a femoral arterial catheter inserted into a leg of the patient.

As illustrated in FIG. 2, hemodynamic sensor 16 includes housing 18, fluid input port 20, catheter-side fluid port 22, and I/O cable 24. Fluid input port 20 is configured to be connected via tubing or other hydraulic connection to a fluid source, such as a saline bag or other fluid input source. Catheter-side fluid port 22 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). I/O cable 24 is configured to connect to hemodynamic monitor 10 via, e.g., one or more of I/O connectors 14 (FIG. 1). Housing 18 of hemodynamic sensor 16 encloses one or more pressure transducers, communication circuitry, processing circuitry, and corresponding electronic components to sense fluid pressure corresponding to arterial pressure of the patient that is transmitted to hemodynamic monitor 10 (FIG. 1) via I/O cable 24.

In operation, a column of fluid (e.g., saline solution) is introduced from a fluid source (e.g., a saline bag) through hemodynamic sensor 16 via fluid input port 20 to catheter-side fluid port 22 toward the catheter inserted into the patient. Arterial pressure is communicated through the fluid column to pressure sensors located within housing 16 which sense the pressure of the fluid column. Hemodynamic sensor 16 translates the sensed pressure of the fluid column to an electrical signal via the pressure transducers and outputs the corresponding electrical signal to hemodynamic monitor 10 (FIG. 1) via I/O cable 24. Hemodynamic sensor 16 therefore transmits analog sensor data (or a digital representation of the analog sensor data) to hemodynamic monitor 10 (FIG. 1) that is representative of substantially continuous beat-to-beat monitoring of the arterial pressure of the patient.

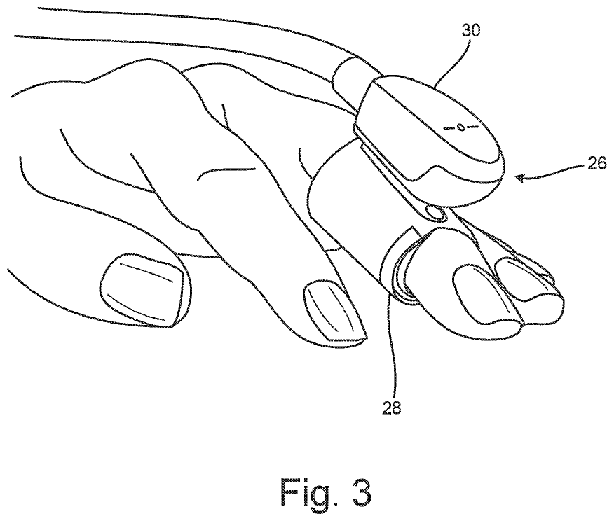

FIG. 3 is a perspective view of hemodynamic sensor 26 for sensing hemodynamic data representative of arterial pressure of a patient. Hemodynamic sensor 26, illustrated in FIG. 3, is one example of a non-invasive hemodynamic sensor that can be attached to the patient via one or more finger cuffs to sense data representative of arterial pressure of the patient. As illustrated in FIG. 3, hemodynamic sensor 26 includes inflatable finger cuff 28 and heart reference sensor 30. Inflatable finger cuff 28 includes an inflatable blood pressure bladder configured to inflate and deflate as controlled by a pressure controller (not illustrated) that is pneumatically connected to inflatable finger cuff 28. Inflatable finger cuff 28 also includes an optical (e.g., infrared) transmitter and an optical receiver that are electrically connected to the pressure controller (not illustrated) to measure the changing volume of the arteries under the cuff in the finger.

In operation, the pressure controller continually adjusts pressure within the finger cuff to maintain a constant volume of the arteries in the finger (i.e., the unloaded volume of the arteries) as measured via the optical transmitter and optical receiver of inflatable finger cuff 28. The pressure applied by the pressure controller to continuously maintain the unloaded volume is representative of the blood pressure in the finger and is communicated by the pressure controller to hemodynamic monitor 10 shown in FIG. 1. Heart reference sensor 30 measures the hydrostatic height difference between the level at which the finger is kept and the reference level for the pressure measurement, which typically is heart level. Accordingly, hemodynamic sensor 26 transmits sensor data that is representative of substantially continuous beat-to-beat monitoring of the arterial pressure waveform of the patient.

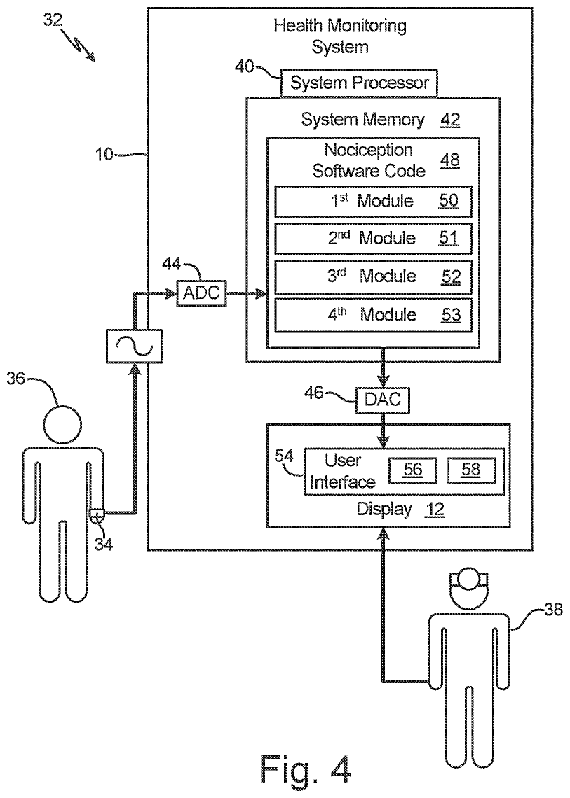

FIG. 4 is a block diagram of hemodynamic monitoring system 32 that determines a nociception score representing a probability of a current nociception event of patient 36 based on a plurality of signal measures derived from the arterial pressure of the patient. Hemodynamic monitoring system 32 monitors the arterial pressure of patient 36 and provides a warning to medical worker 38 when the nociception score of patient 36 rises above a predetermined threshold. Medical worker 38 can respond to the warning by administering an appropriate analgesic to patient 36 to mitigate the current nociception event.

As illustrated in FIG. 4, hemodynamic monitoring system 32 includes hemodynamic monitor 10 and hemodynamic sensor 34. Hemodynamic monitoring system 32 can be implemented within a patient care environment, such as an ICU, an OR, or other patient care environment. As illustrated in FIG. 4, the patient care environment can include patient 36 and healthcare worker 38 trained to utilize hemodynamic monitoring system 32.

Hemodynamic monitor 10, as described above with respect to FIG. 1, can be, e.g., an integrated hardware unit including system processor 40, system memory 42, display 12, analog-to-digital (ADC) converter 44, and digital-to-analog (DAC) converter 46. In other examples, any one or more components and/or described functionality of hemodynamic monitor 10 can be distributed among multiple hardware units. For instance, in some examples, display 12 can be a separate display device that is remote from and operatively coupled with hemodynamic monitor 10. In general, though illustrated and described in the example of FIG. 4 as an integrated hardware unit, it should be understood that hemodynamic monitor 10 can include any combination of devices and components that are electrically, communicatively, or otherwise operatively connected to perform functionality attributed herein to hemodynamic monitor 10.

As illustrated in FIG. 4, system memory 42 stores nociception software code 48 which forms the detection model of hemodynamic monitor 10. Nociception software code 48 includes first module 50 for extracting and calculating waveform features and a plurality of signal measures from the arterial pressure of patient 36, second module 51 for organizing the plurality of signal measures into pairs of signal measures, third module 52 for measuring a cross-correlational association of each of the pairs of signal measures, and fourth module 53 for monitoring the measured cross-correlational association of each of the pairs of signal measures for a burst (i.e., an increase above a predetermined threshold) in the measured cross-correlational association of at least one of the pairs of signal measures.

Display 12 provides user interface 54, which includes control elements 56 that enable user interaction with hemodynamic monitor 10 and/or other components of hemodynamic monitoring system 32. User interface 54, as illustrated in FIG. 4, also provides sensory alarm 58 to provide warning to medical personnel of a current nociception event of patient 36, as is further described below. Sensory alarm 58 can be implemented as one or more of a visual alarm, an audible alarm, a haptic alarm, or other type of sensory alarm. For instance, sensory alarm 58 can be invoked as any combination of flashing and/or colored graphics shown by user interface 54 on display 12, display of the nociception score via user interface 54 on display 12, a warning sound such as a siren or repeated tone, and a haptic alarm configured to cause hemodynamic monitor 10 to vibrate or otherwise deliver a physical impulse perceptible to medical worker 38 or other user.

Hemodynamic sensor 34 can be attached to patient 36 to sense hemodynamic data representative of the arterial pressure waveform of patient 36. Hemodynamic sensor 34 is operatively connected to hemodynamic monitor 10 (e.g., electrically and/or communicatively connected via wired or wireless connection, or both) to provide the sensed hemodynamic data to hemodynamic monitor 10. In some examples, hemodynamic sensor 34 provides the hemodynamic data representative of the arterial pressure waveform of patient 36 to hemodynamic monitor 10 as an analog signal, which is converted by ADC 44 to digital hemodynamic data representative of the arterial pressure waveform. In other examples, hemodynamic sensor 34 can provide the sensed hemodynamic data to hemodynamic monitor 10 in digital form, in which case hemodynamic monitor 10 may not include or utilize ADC 44. In yet other examples, hemodynamic sensor 34 can provide the hemodynamic data representative of the arterial pressure waveform of patient 36 to hemodynamic monitor 10 as an analog signal, which is analyzed in its analog form by hemodynamic monitor 10.

Hemodynamic sensor 34 can be a non-invasive or minimally invasive sensor attached to patient 36. For instance, hemodynamic sensor 34 can take the form of minimally invasive hemodynamic sensor 16 (FIG. 2), non-invasive hemodynamic sensor 26 (FIG. 3), or other minimally invasive or non-invasive hemodynamic sensor. In some examples, hemodynamic sensor 34 can be attached non-invasively at an extremity of patient 36, such as a wrist, an arm, a finger, an ankle, a toc, or other extremity of patient 36. As such, hemodynamic sensor 34 can take the form of a small, lightweight, and comfortable hemodynamic sensor suitable for extended wear by patient 36 to provide substantially continuous beat-to-beat monitoring of the arterial pressure of patient 36 over an extended period of time, such as minutes or hours.

In certain examples, hemodynamic sensor 34 can be configured to sense an arterial pressure of patient 36 in a minimally invasive manner. For instance, hemodynamic sensor 34 can be attached to patient 36 via a radial arterial catheter inserted into an arm of patient 36. In other examples, hemodynamic sensor 34 can be attached to patient 36 via a femoral arterial catheter inserted into a leg of patient 36. Such minimally invasive techniques can similarly enable hemodynamic sensor 34 to provide substantially continuous beat-to-beat monitoring of the arterial pressure of patient 36 over an extended period of time, such as minutes or hours.

System processor 40 is a hardware processor configured to execute nociception software code 48, which implements first module 50, second module 51, third module 52, and fourth module 53 to produce a nociception score representing a probability of a current nociception event for patient 36. Examples of system processor 40 can include any one or more of a microprocessor, a controller, a digital signal processor (DSP), an application specific integrated circuit (ASIC), a field-programmable gate array (FPGA), or other equivalent discrete or integrated logic circuitry.

System memory 42 can be configured to store information within hemodynamic monitor 10 during operation. System memory 42, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 42 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

Display 12 can be a liquid crystal display (LCD), a light-emitting diode (LED) display, an organic light-emitting diode (OLED) display, or other display device suitable for providing information to users in graphical form. User interface 54 can include graphical and/or physical control elements that enable user input to interact with hemodynamic monitor 10 and/or other components of hemodynamic monitoring system 32. In some examples, user interface 54 can take the form of a graphical user interface (GUI) that presents graphical control elements presented at, e.g., a touch-sensitive and/or presence sensitive display screen of display 12. In such examples, user input can be received in the form of gesture input, such as touch gestures, scroll gestures, zoom gestures, or other gesture input. In certain examples, user interface 54 can take the form of and/or include physical control elements, such as a physical buttons, keys, knobs, or other physical control elements configured to receive user input to interact with components of hemodynamic monitoring system 32. User interface 54 can include a speaker that allows hemodynamic monitor 10 the ability to generate an audible alarm.

In operation, hemodynamic sensor 34 senses hemodynamic data representative of an arterial pressure waveform of patient 36. Hemodynamic sensor 34 provides the hemodynamic data (e.g., as analog sensor data), to hemodynamic monitor 10. ADC 44 converts the analog hemodynamic data to digital hemodynamic data representative of the arterial pressure waveform of patient 36.

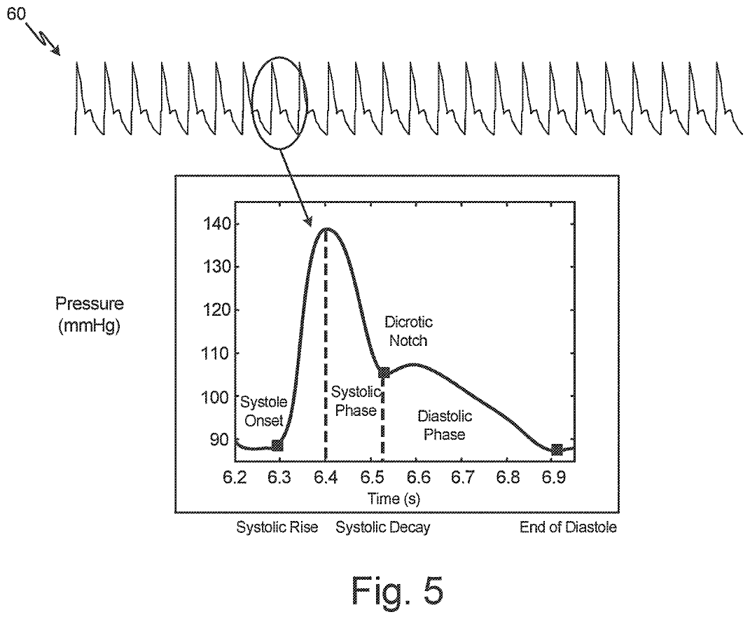

System processor 40 executes nociception software code 48 to determine, using the received hemodynamic data, a nociception detection score representing a probability of a current nociception event for patient 36. For instance, system processor 40 can execute first module 50 to perform waveform analysis of the hemodynamic data to determine the plurality of signal measures. System processor 40 executes second module 51 to organize the plurality of signal measures into pairs of signal measures. System processor 40 executes third module 52 to measure or calculate the cross-correlational association of each pair of the signal measures. System processor 40 then executes fourth module 53 to monitor the measured cross-correlational association of each of the pairs of signal measures over time. As fourth module 53 monitors the measured cross-correlational association of each of the pairs of signal measures, fourth module 53 can output a plot of the measured cross-correlational association of each of the pairs of signal measures over time to display 12 to allow medical worker 38 to view the values of the measured cross-correlational association of each of the pairs of signal measures. If fourth module 53 detects a burst (i.e., an increase above a predetermined threshold) in the measured cross-correlational association of at least one of the pairs of signal measures, system processor 40 invokes sensory alarm 58 of user interface 54 to send a sensory signal to alert medical worker 38 that patient 36 is presently experiencing a current nociception event. Medical worker 38 can respond to the warning by administering an analgesic to patient 36, or administering any other form of treatment to patient 36, to mitigate the current nociception event. How first module 50 performs the waveform analysis of the hemodynamic data to determine the plurality of signal measures is discussed below in greater detail with reference to FIG. 5.

FIG. 5 provides an example graph illustrating an example trace of an arterial pressure waveform of patient 36 with an individual cardiac cycle identified and enlarged. Performing waveform analysis of the hemodynamic data of patient 36 sensed by hemodynamic sensor 34 can include identifying individual cardiac cycles in the arterial pressure waveform of patient 36 and identifying a dicrotic notch in each of the individual cardiac cycles of each of the arterial pressure waveforms of the sensed hemodynamic data, similar to the example shown in FIG. 5. Next, the waveform analysis on the sensed hemodynamic data includes identifying a systolic rise phase, a systolic decay phase, and a diastolic phase in each of the individual cardiac cycles of each of the arterial pressure waveforms of the sensed hemodynamic data, similar to the example shown in FIG. 5.

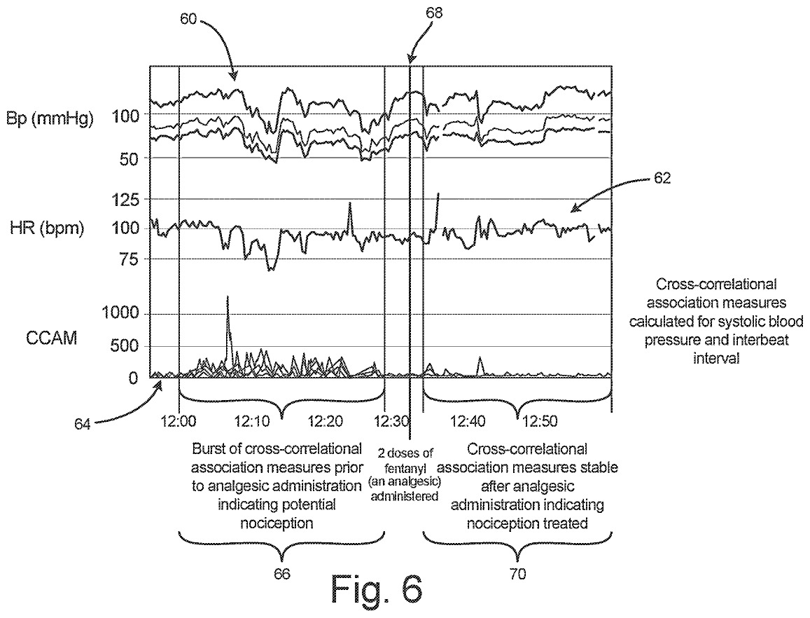

Signal measures are extracted from each of the systolic rise phase, the systolic decay phase, and the diastolic phase from each of the individual cardiac cycles of each of the arterial pressure waveforms of patient 36 sensed by hemodynamic sensor 34. The signal measures can correspond to hemodynamic effects from each of the systolic rise phase, the systolic decay phase, and the diastolic phase from each of the individual cardiac cycles. Those hemodynamic effects can include contractility, aortic compliance, stroke volume, vascular tone, afterload, and full cardiac cycle, all of which can be impacted by the sympathetic nervous response triggered by the nociception event. The signal measures calculated or extracted by the waveform analysis include a mean, a maximum, a minimum, a duration, an area, a standard deviation, derivatives, and/or morphological measures from each of the systolic rise phase, the systolic decay phase, and the diastolic phase from each of the individual cardiac cycles. The signal measures can also include heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles of each of the arterial pressure waveforms of patient 36 sensed by hemodynamic sensor 34. As noted above with reference to FIG. 4, system processor 40 can execute first module 50 to perform waveform analysis of the hemodynamic data to determine the plurality of signal measures. After the plurality of signal measures are determined, hemodynamic monitor 10 organizes the plurality of signal measures into pairs and calculates the cross-correlational association measure (CCAM) of each pair of signal measures, as described below with reference to FIG. 6.

FIG. 6 is a graph illustrating a plot of systolic, diastolic, and mean blood pressure 60, a plot of heart rate 62, and a collection of cross-correlational association measure (CCAM) plots 64 all plotted over time for patient 36. FIG. 6 also shows burst 66 in the CCAM plots 64, a drug administration event 68 in which analgesic medication was administered (phentanyl), and stable period 70 in the CCAM plots 64. The graph of FIG. 6 can be an example of what is outputted by fourth module 53 of nociception software code 48 and shown on display 12 of hemodynamic monitor 10. Systolic blood pressure 60 and heart rate 62 are signal measures derived from performing waveform analysis of the hemodynamic data of patient 36 sensed by hemodynamic sensor 34 (shown and discussed above with regards to FIGS. 4 and 5). Hemodynamic monitor 10 calculates a cross-correlational association measure between systolic blood pressure 60 and heart rate 62 and outputs the cross-correlational association measure as shown in the CCAM plot 64. Hemodynamic monitor 10 can calculate the cross-correlational association measure (CCAM) between systolic blood pressure 60 and heart rate 62 by taking a ratio of the standard deviation of heart rate 62 and the standard deviation of systolic blood pressure 60, represented by the equation below:

$$C C A M = \frac{SD ( y )}{SD ( x )} \qquad ( Equation 1 )$$

where y is heart rate 62, x is systolic blood pressure 60, SD (y) is the standard deviation of heart rate 62, SD (x) is the standard deviation systolic blood pressure 60, and CCAM is the cross-correlational association measure of heart rate 62 and systolic pressure 60. The values for CCAM are then plotted over time as CCAM plots 64. Equation 1 can be applied to all other pairs of the signal measures that are extracted from each of the systolic rise phase, the systolic decay phase, the diastolic phase and the entire waveform from each of the individual cardiac cycles on FIG. 5 to derive additional cross-correlational association plots that are all congregated together as CCAM plots 64. The CCAM plots 64 of each of the pairs of signal measures can be normalized before outputting to display 12 to allow all of the measured CCAM for the pairs of signal measures to fit on display 12 and overlay one another.

The CCAM plots 64 are monitored for bursts 66 by system processor 40 of hemodynamic monitor 10. Bursts 66 occur in CCAM plots 64 when at least one of the plots in the CCAM plots 64 increases above a predetermined threshold. The monitoring/detection of bursts 66 can be performed using a wide variety of mathematical methods, such as detection of amplitude change, change in the area under the curve of each burst 66, and/or changes in the standard deviation of each burst 66. The monitoring/detection of bursts 66 can also be performed with more complex time and frequency domain methods, such as Fast Fourier transform (FFT) based methods and wavelet-based methods. In one example, the predetermined threshold is 50% greater than a moving average of the CCAM plots 64. Thus, in this example, a burst 66 occurs when at least one of the CCAM plots 64 is at least 50% greater than the moving average of the CCAM plots 64 of the pairs of signal measures. If a burst 66 occurs in CCAM for systolic blood pressure 60 and heart rate 62, as shown in the CCAM plots 64 of FIG. 6, hemodynamic monitor 10 interprets that burst 66 as a current nociception event of patient 36 and sends an alert to medical personnel. In response to the alert, medical personnel can administer an analgesic to patient 36, represented by drug administration event 68 in FIG. 6, to alleviate the nociception of patient 36. If patient 36 responds to the analgesic, burst 66 should dissipate below the threshold in CCAM plots 64 and be followed by stable period 70. During stable period 70, none of CCAM plots 64 of the signal measures exceed the threshold, indicating that patient 36 is not experiencing nociception.

While FIG. 6 has been discussed with regards to the signal measure pair of systolic blood pressure 60 and heart rate 62, additional signal measure pairs will be analyzed by hemodynamic monitor 10 and the cross-correlational association measures of those signal measure pairs will be calculated and included in CCAM plots 64. In some examples, hemodynamic monitor 10 can monitor and plot all possible combinations of any two signal measures derived from the sensed hemodynamic data representative of the arterial pressure waveform of patient 36, which can include thousands and tens of thousands of signal pairs and their corresponding cross-correlational association measures. Nociception will be manifested in different patients by different signal measures from their sensed hemodynamic data. Configuring hemodynamic monitor 10 to plot and monitor cross-correlational association measures for as many combinations of signal measures as possible allows hemodynamic monitor 10 to detect nociception in different patients.

While the example of FIG. 6 calculates cross-correlational association measures of the signal measure pairs using Equation 1, in other examples, the CCAM of systolic blood pressure 60 and heart rate 62 can be measured by calculating the product between signal measures, or calculating a ratio of the signal measures, or any known mathematical method for calculating cross-correlational associations between two variables. Hemodynamic monitor 10 can be programmed to use more than one equation or method to calculate cross-correlational associations for pairs of signal measures.

In other examples, hemodynamic monitor 10 can be programmed to calculate a generalized cross correlation (GGC) between a pair of signal measures before calculating the CCAM of the pair of signal measures. Hemodynamic monitor 10 can calculate the GGC between the pair of signal measures by first measuring a ten second window for both a first signal measure (such as systolic blood pressure) and a second signal measure (such as heart rate), represented by the equation below:

$$G G C = {Σ}_{m = 1}^{10} x [ m ] y [ m + τ ] , where τ = 0 , 1 , 2 , … , 5 \qquad ( Equation 2 )$$

where m is a time window measured in seconds, x is the first signal measure (such as systolic blood pressure), y is the second signal measure (such as heart rate), t is a time shift, and GGC is the generalized cross correlation between the first signal measure and the second signal measure. As shown in Equation 2, the first signal measure and the second signal measure are both interpolated and resampled within a ten second window at one second intervals. The ten second window of the first signal measure is cross-correlated with the ten second window of the second signal measure. The time shift t is applied to one of the signal measures with the cross-correlation repeated to achieve a pattern of cross-correlations that include a maximum cross-correlation between the first signal measure and the second signal measure. The maximum cross-correlation (or maximum GGC) indicates the best time delay between the first signal measure and the second signal measure to achieve the highest cross-correlation between the first signal measure and the second signal measure. If the highest cross-correlation is significant at a p-value (the probability that the null hypothesis is true) that is less than 0.05 or any other threshold, Equation 1 can then be applied to the first signal measure and the second signal measure at the best time delay to obtain the CCAM for the first signal measure and the second signal measure. Equation 2 and Equation 1 can be applied to other pairs of signal measures to derive additional plots that are all congregated together as CCAM plots 64. While the example above discloses a time window m of ten seconds, the time window m can be less than ten seconds or greater than ten seconds. Also, while the example above discloses a time shift t of zero to five seconds with a resampling interval of one second, the time shift t can be of set of delays (such as zero to ten seconds in length or negative 10 to positive 10 seconds in length), and the resampling interval can be in any time increment (such as 0.1 second, 0.5 second, 1 second, or 2 seconds). For example, the resampling interval can be any value from 0.1 second to 2 seconds.

### Discussion of Possible Embodiments

The following are non-exclusive descriptions of possible embodiments of the present invention.

A method for monitoring arterial pressure of a patient and detecting nociception of the patient, the method comprising: receiving, by a hemodynamic monitor, sensed hemodynamic data representative of an arterial pressure waveform of the patient from a hemodynamic sensor; performing, by a hardware processor of the hemodynamic monitor, waveform analysis of the sensed hemodynamic data to calculate a plurality of signal measures of the sensed hemodynamic data; organizing, by the hardware processor of the hemodynamic monitor, the plurality of signal measures into pairs of signal measures; measuring, by the hardware processor of the hemodynamic monitor, a cross-correlational association of each of the pairs of signal measures; outputting to a display a measured cross-correlational association of each of the pairs of signal measures; monitoring the measured cross-correlational association of each of the pairs of signal measures for an increase above a predetermined threshold in the measured cross-correlational association of at least one of the pairs of signal measures; and sending, by the hemodynamic monitor, an alert to medical personnel of a nociception event of the patient when the measured cross-correlational association of at least one of the pairs of signal measures increases above the predetermined threshold.

The method of the preceding paragraph can optionally include, additionally and/or alternatively, any one or more of the following features, configurations and/or additional components:

The predetermined threshold is at least 50% greater than a moving average of the measured cross-correlational association of the at least one of the pairs of signal measures.

Normalizing the measured cross-correlational association of each of the pairs of signal measures before outputting the measured cross-correlational association of each of the pairs of signal measures to the display.

The signal measures comprise hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

The signal measures comprise a mean, a maximum, a minimum, a duration, an area, a standard deviation, derivatives, and/or morphological measures from each of the systolic rise phase, the systolic decay phase, and the diastolic phase from each of the individual cardiac cycles.

The signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

Measuring the cross-correlational association of each of the pairs of signal measures by the hardware processor comprises: measuring a time window of data for each of the signal measures; interpolating each of the signal measures and resampling each of the signal measures at one second intervals; calculating a cross correlation value between the pair of the signal measures at a set of delays; selecting the delay that provides the highest cross correlation value; measuring a standard deviation of a first signal measure at the delay that provides the highest cross correlation value; measuring a standard deviation of a second signal measure at the delay that provides the highest cross correlation value; and calculating a ratio between the standard deviation of the first signal measure and the standard deviation of the second signal measure if the highest cross correlation value has a p-value less than 0.05.

The time window comprises ten seconds.

The time window is greater than ten seconds.

The time window is less than ten seconds.

The set of delays is from zero to ten seconds with a resampling interval of one second.

The set of delays is from negative ten to positive ten seconds with a resampling interval of 0.1 to 2 seconds.

The set of delays is from zero to five seconds with a resampling interval of 0.1 to 2 seconds.

A method for monitoring arterial pressure of a patient and detecting nociception of the patient, the method comprising: receiving, by a hemodynamic monitor, sensed hemodynamic data representative of an arterial pressure waveform of the patient; performing, by a hardware processor of the hemodynamic monitor, waveform analysis of the sensed hemodynamic data to calculate a plurality of signal measures of the sensed hemodynamic data; calculating, by the hardware processor of the hemodynamic monitor, cross-correlational association measurements between each of the signal measures; outputting to a user interface the cross-correlational association measurements; monitoring the cross-correlational association measurements for bursts in the cross-correlational association measurements; and detecting a nociception event of the patient when a burst in one or more of the cross-correlational association measurements is outputted to the user interface.

The method of the preceding paragraph can optionally include, additionally and/or alternatively, any one or more of the following features, configurations and/or additional components:

Performing waveform analysis of the sensed hemodynamic data to calculate the plurality of signal measures of the sensed hemodynamic data comprises: identifying individual cardiac cycles in the arterial pressure waveform; identifying a dicrotic notch in each of the individual cardiac cycles; identifying a systolic rise phase, a systolic decay phase, and a diastolic phase in each of the individual cardiac cycles; and extracting the plurality of signal measures from each of the systolic rise phase, the systolic decay phase, and the diastolic phase from each of the individual cardiac cycles.

The plurality of signal measures comprises hemodynamic effects from each of the systolic rise phase, the systolic decay phase, and the diastolic phase from each of the individual cardiac cycles, wherein the hemodynamic effects comprise contractility, aortic compliance, stroke volume, vascular tone, afterload, and full cardiac cycle.

The plurality of signal measures comprises a mean, a maximum, a minimum, a duration, an area, a standard deviation, derivatives, and/or morphological measures from each of the systolic rise phase, the systolic decay phase, and the diastolic phase from each of the individual cardiac cycles.

The plurality of signal measures comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

The user interface comprises at least one of a display screen and a speaker.

A system for monitoring arterial pressure of a patient and providing a warning to medical personnel of nociception of the patient, the system comprising: a hemodynamic sensor that produces hemodynamic data representative of an arterial pressure waveform of the patient; a system memory that stores nociception detection software code; a user interface that includes a sensory alarm that provides a sensory signal to warn the medical personnel of a nociception event of the patient; and a hardware processor that is configured to execute the nociception detection software code to: perform waveform analysis of the hemodynamic data to determine a plurality of signal measures; organize the plurality of signal measures into pairs of signal measures; measure a cross-correlational association of each of the pairs of signal measures; output to the user interface measurements of cross-correlational association of the pairs of signal measures; monitor the measurements of cross-correlational association of the pairs of signal measures for bursts in the measurements of cross-correlational association of the pairs of signal measures; and invoke a sensory alarm of the user interface indicating a nociception event in response to a burst in one or more of the measurements of cross-correlational association of the pairs of signal measures.

The system of the preceding paragraph can optionally include, additionally and/or alternatively, any one or more of the following features, configurations and/or additional components:

The hemodynamic sensor is a noninvasive hemodynamic sensor that is attachable to an extremity of the patient.

The hemodynamic sensor is a minimally invasive arterial catheter based hemodynamic sensor.

The hemodynamic sensor produces the hemodynamic data as an analog hemodynamic sensor signal representative of the arterial pressure waveform of the patient.

An analog-to-digital converter that converts the analog hemodynamic sensor signal to digital hemodynamic data representative of the arterial pressure waveform of the patient.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed, but that the invention will include all embodiments falling within the scope of the appended claims.

1. A method for monitoring arterial pressure of a patient and detecting nociception of the patient, the method comprising:
receiving, by a hemodynamic monitor, sensed hemodynamic data representative of an arterial pressure waveform of the patient from a hemodynamic sensor;
performing, by a hardware processor of the hemodynamic monitor, waveform analysis of the sensed hemodynamic data to calculate a plurality of signal measures of the sensed hemodynamic data;
organizing, by the hardware processor of the hemodynamic monitor, the plurality of signal measures into pairs of signal measures;
measuring, by the hardware processor of the hemodynamic monitor, a cross-correlational association of each of the pairs of signal measures;
outputting to a display a measured cross-correlational association of each of the pairs of signal measures;
monitoring the measured cross-correlational association of each of the pairs of signal measures for an increase above a predetermined threshold in the measured cross-correlational association of at least one of the pairs of signal measures; and
sending, by the hemodynamic monitor, an alert to medical personnel of a nociception event of the patient when the measured cross-correlational association of at least one of the pairs of signal measures increases above the predetermined threshold.

2. The method of claim 1, wherein the predetermined threshold is at least 50% greater than a moving average of the measured cross-correlational association of the at least one of the pairs of signal measures.

3. The method of claim 2, further comprising normalizing the measured cross-correlational association of each of the pairs of signal measures before outputting the measured cross-correlational association of each of the pairs of signal measures to the display.

4. The method of claim 3, wherein the signal measures comprise hemodynamic effects from each of the systolic rise phase, the systolic decay phase, and the diastolic phase from each of the individual cardiac cycles, wherein the hemodynamic effects comprise contractility, aortic compliance, stroke volume, vascular tone, afterload, and full cardiac cycle, and/or wherein the signal measures comprise a mean, a maximum, a minimum, a duration, an area, a standard deviation, derivatives, and/or morphological measures from each of the systolic rise phase, the systolic decay phase, and the diastolic phase from each of the individual cardiac cycles, and/or the signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

5. The method of claim 4, wherein measuring the cross-correlational association of each of the pairs of signal measures by the hardware processor comprises:
measuring a time window of data for each of the signal measures;
interpolating each of the signal measures and resampling each of the signal measures at one second intervals;
calculating a cross correlation value between the pair of the signal measures at a set of delays;
selecting the delay that provides the highest cross correlation value;
measuring a standard deviation of a first signal measure at the delay that provides the highest cross correlation value;
measuring a standard deviation of a second signal measure at the delay that provides the highest cross correlation value; and
calculating a ratio between the standard deviation of the first signal measure and the standard deviation of the second signal measure if the highest cross correlation value has a p-value less than 0.05.

6. The method of claim 5, wherein the time window comprises ten seconds.

7. The method of claim 5, wherein the time window is greater than ten seconds.

8. The method of claim 5, wherein the time window is less than ten seconds.

9. The method of claim 5, wherein the set of delays is from zero to ten seconds with a resampling interval of one second.

10. The method of claim 5, wherein the set of delays is from negative ten to positive ten seconds with a resampling interval of 0.1 to 2 seconds.

11. The method of claim 5, wherein the set of delays is from zero to five seconds with a resampling interval of 0.1 to 2 seconds.

12. A method for monitoring arterial pressure of a patient and detecting nociception of the patient, the method comprising:
receiving, by a hemodynamic monitor, sensed hemodynamic data representative of an arterial pressure waveform of the patient;
performing, by a hardware processor of the hemodynamic monitor, waveform analysis of the sensed hemodynamic data to calculate a plurality of signal measures of the sensed hemodynamic data;
calculating, by the hardware processor of the hemodynamic monitor, cross-correlational association measurements between each of the signal measures;
outputting to a user interface the cross-correlational association measurements;
monitoring the cross-correlational association measurements for bursts in the cross-correlational association measurements; and
detecting a nociception event of the patient when a burst in one or more of the cross-correlational association measurements is outputted to the user interface.

13. The method of claim 12, wherein performing waveform analysis of the sensed hemodynamic data to calculate the plurality of signal measures of the sensed hemodynamic data comprises:
identifying individual cardiac cycles in the arterial pressure waveform;
identifying a dicrotic notch in each of the individual cardiac cycles;
identifying a systolic rise phase, a systolic decay phase, and a diastolic phase in each of the individual cardiac cycles; and
extracting the plurality of signal measures from each of the systolic rise phase, the systolic decay phase, and the diastolic phase from each of the individual cardiac cycles.

14. The method of claim 13, wherein the plurality of signal measures comprises hemodynamic effects from each of the systolic rise phase, the systolic decay phase, and the diastolic phase from each of the individual cardiac cycles, wherein the hemodynamic effects comprise contractility, aortic compliance, stroke volume, vascular tone, afterload, and full cardiac cycle, and wherein the plurality of signal measures comprises a mean, a maximum, a minimum, a duration, an area, a standard deviation, derivatives, and/or morphological measures from each of the systolic rise phase, the systolic decay phase, and the diastolic phase from each of the individual cardiac cycles and/or the plurality of signal measures comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

15. The method of claim 12, wherein the user interface comprises at least one of a display screen and a speaker.

16. A system for monitoring arterial pressure of a patient and providing a warning to medical personnel of nociception of the patient, the system comprising:
a hemodynamic sensor that produces hemodynamic data representative of an arterial pressure waveform of the patient;
a system memory that stores nociception detection software code;
a user interface that includes a sensory alarm that provides a sensory signal to warn the medical personnel of a nociception event of the patient; and
a hardware processor that is configured to execute the nociception detection software code to:
perform waveform analysis of the hemodynamic data to determine a plurality of signal measures;
organize the plurality of signal measures into pairs of signal measures;
measure a cross-correlational association of each of the pairs of signal measures;
output to the user interface measurements of cross-correlational association of the pairs of signal measures;
monitor the measurements of cross-correlational association of the pairs of signal measures for bursts in the measurements of cross-correlational association of the pairs of signal measures; and
invoke a sensory alarm of the user interface indicating a nociception event in response to a burst in one or more of the measurements of cross-correlational association of the pairs of signal measures.

17. The system of claim 16, wherein the hemodynamic sensor is a non-invasive hemodynamic sensor that is attachable to an extremity of the patient.

18. The system of claim 16, wherein the hemodynamic sensor is a minimally invasive arterial catheter based hemodynamic sensor.

19. The system of any of claim 16, wherein the hemodynamic sensor produces the hemodynamic data as an analog hemodynamic sensor signal representative of the arterial pressure waveform of the patient.

20. The system of claim 19, and further comprising an analog-to-digital converter that converts the analog hemodynamic sensor signal to digital hemodynamic data representative of the arterial pressure waveform of the patient.
